(19) United States
(12) Patent Application Publication
(10) Pub. No.: US 2007/0146631 A1
(43) Pub. Date: Jun. 28, 2007

(54) System and method for analysis and visualization of metamorphopsia through three dimensional scene regeneration and testing of vision thereby

(76) Inventors: Stephen Sinclair, Gladwyn, PA (US); Peter Presti, Duluth, GA (US)

Correspondence Address: Lawrence A. Husick;Lipton, Weinberger & Husick, 201 North Jackson Street, Media, PA, 19063, US

(21) Appl. No.: 11/318,085

(22) Filed: Dec. 24, 2005

(51) Int. Cl. A61B 3/00

(52) U.S. Cl. 351/200

(57) ABSTRACT

With distortion data that is derived from an interactive test of a patient, the monocular two dimensional, binocular two dimensional and three dimensional environment may be reconstructed from the patient's point of view. This process involves projection of the three dimensional dataset onto each binocular two dimensional retinal plane. The planar data is perturbed by the documented distortions and two thee dimensional datasets are reconstructed. Visualization of the reconstructed datasets allows a third party, such as an ophthalmologist or family member to observe the perturbations that the patient sees.

This application is a continuation in part of an provisional application Ser. No. 60/080,061, filed Mar. 31, 1998 by the inventors hereof.

### BACKGROUND OF THE INVENTION

Metamorphopsia is a vision defect caused by a distorted retina in one or both eyes. The inflicted patient perceives this condition as a dynamic distortion of the geometry of his environment. Metamorphopsia transforms the observed world into a perpetual Escher-like environment. Mild horizontal distortions produce binocular depth perception errors. More severe horizontal distortions as well as vertical distortions cause ghosting or frank double vision. The patient finds navigating stairs or non-uniform topologies a challenge. The sudden onset of the distortion causes the greatest difficulties, whereas if the distortion develops more slowly there may be some adaptation to the sensory input such that the patient may not even realize that a visual defect exists.

### SUMMARY OF THE INVENTION

A tool to document and quantify metamorphopsia has been developed as an extension of the Multi-Axis Vision Evaluation System (MAVES) of Sinclair. With distortion data that is derived from an interactive test of a patient with MAVES, the monocular two dimensional, and binocular two dimensional and three dimensional environment may be reconstructed from the patient's point of view. This process involves projection of the three dimensional dataset onto each binocular two dimensional retinal plane. The planar data is perturbed by the documented distortions and two thee dimensional datasets are reconstructed. Visualization of the reconstructed datasets allows a third party, such as an ophthalmologist or family member to observe the perturbations that the patient sees.

The MAVES testing apparatus preferably comprises a 21″ video monitor and a personal computer, a chinrest to fix the patient's head, and interaction control pad, and a fixation monitoring device, typically an infrared pupil tracker. The metamorphopsia analysis is preferably conducted using a computer program that presents 5×5 regular spline grid with random perturbations overlaid on a cross, which the patient fixates at its center. The display grid dims if the pupil tracker detects a fixation deviation. The patient is instructed to remove the distortions from the grid until all of the lines are straight and uniform. He accomplishes this by manipulating the vertices of the grid using a pointing device and selection controls. The resulting grid objectively quantifies the complement of macular distortions perceived by the patient.

For patients with severe vision defects, the spline grid may be constructed on a line by line basis, as well as in subsections or quadrants. In addition, the fineness of the spline grid may be increased to permit microdistortions to be recognized and mapped.

A two dimensional monocular or binocular reconstruction by applying the complementary distortions derived from a “corrected” spline grid may be constructed by conventional graphical processing means to aid in illustrating the patient's perceptions.

A three dimensional environment may be reconstructed to demonstrate what is perceived by the patient under varying binocular conditions. This process comprises projecting the three dimensional point coordinates of the data set onto each binocular two dimensional retinal plane through each eye's nodal point. The two dimensional planar points are perturbed by the documented distortion measured for each eye. Two vectors defined by the perturbed two dimensional points in each retinal plane and corresponding nodal point are projected back into the world space occupied by the original geometry to create two new three dimensional geometry datasets. The position where the two vectors are closest is analyzed. If the distance between the vectors (d) is smaller that a convergence error limit (k) then the center point of the line drawn between the two vectors defines the new three dimensional point for both geometry datasets. If d>k then two new three dimensional points are defined by the closest point of approach for each vector.

The two three dimensional geometry sets are rendered to produce two images, on for each eye of the patient. A composite of both images is generated with a default 50% transparency (adjusted to compensate for eye dominance, if known) to produce a single image representative of the patient's perceived environment. As the patient moves, new geometry sets are calculated, rendered, and composited to produce a series of video images.

The warped three dimensional geometry, especially when presented as a short video during ocular or head movement, appears to realistically represent the visual disturbances which these patients describe, and for the first time may provide clinicians an insight into this formerly poorly understood and appreciated visual defect.

### DESCRIPTION OF THE PRIOR ART

The vision evaluation systems of the prior art have analyzed central visual acuity, visual field, high and low contrast vision, and the like, but have not been adapted to quantify visual field distortions which are characteristic of metamorphopsia.

### BRIEF DESCRIPTION OF THE FIGURES

FIG. 1 depicts binocular depth perception errors that result from mild horizontal distortions in the visual system.

FIG. 2a depicts a manipulated spline grid, which has been adjusted to appear regular by a patient suffering from metamorphopsia.

FIG. 2b depicts a graphic of several building facades that has been processed by the method of the present invention to present to an observer with normal (undistorted) vision the perceived monocular image of a patient suffering from metamorphopsia.

FIG. 3a depicts a 70:30 L:R dominance (left eye dominant) binocular image constructed by the two dimensional version of the method of the present invention from spline grid data manipulated by a patient suffering from metamorphopsia.

FIG. 3b depicts a 30:70 L:R dominance (left eye dominant) binocular image constructed by the two dimensional version of the method of the present invention from spline grid data manipulated by a patient suffering from metamorphopsia.

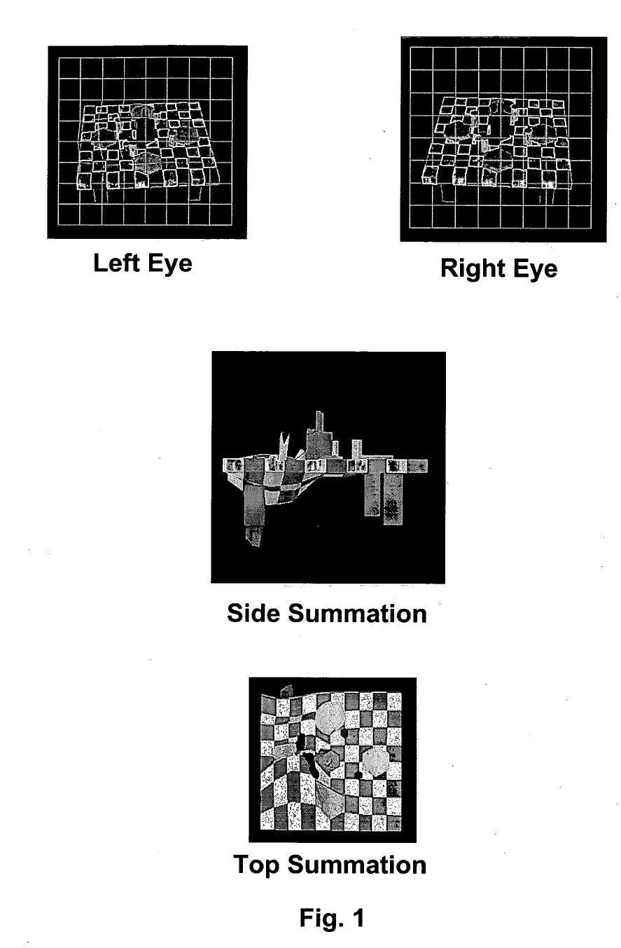

### DETAILED DESCRIPTION OF THE INVENTION

In order to more fully understand the effects of metamorphopsia, we reconstruct a virtual environment as perceived by an afflicted person. This involves documenting the patient's retinal warp and then rendering the virtual environment as perceived by the patient. The result is a warped 3D geometry that reflects depth perception errors due to the inconsistent stereo reconstruction of warped imagery.

Definitions:

World Space—3D space in which the Scene Geometry and Observer lie.
Scene Geometry—Original 3D geometry of the scene. The virtual environment.
Left Eye Geometry—Resulting warped 3D geometry for the left eye.
Right Eye Geometry—Resulting warped 3D geometry for the right eye.
Observer—The Scene Geometry observer that represents the patient. Located in world space. This observer is composed of 2 cameras representing the patient's 2 eyes. For the purposes of this exercise, the cameras lie in the X plane. Up in a camera view is in the direction of the Y axis. The cameras are oriented in the Z direction. These cameras are separated by an X-distance representative of the interocular separation of the patient's eyes.

Eye Nodal Point—3D point representing the nodal point of the Observer's camera. The projection reference point of the Retina Image Plane.

Retina Image Plane—2D plane representing the retina. The image plane for the Observer's cameras.

Method:

Acquire Patient Data

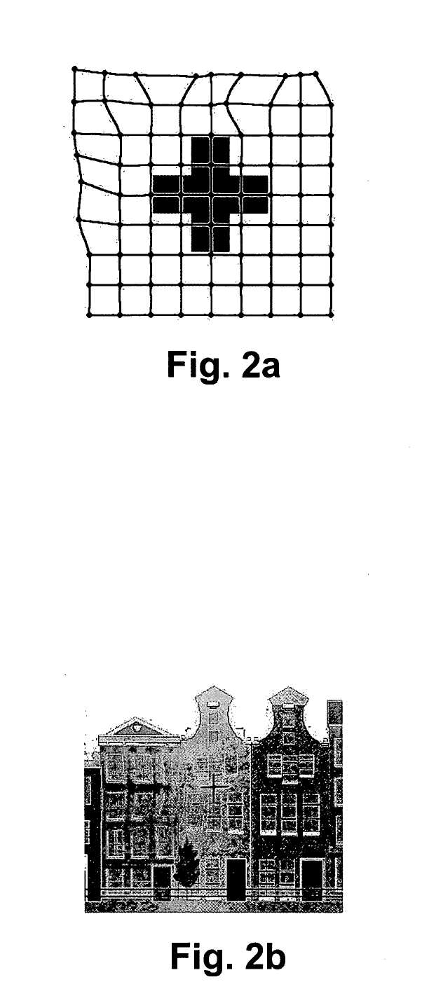

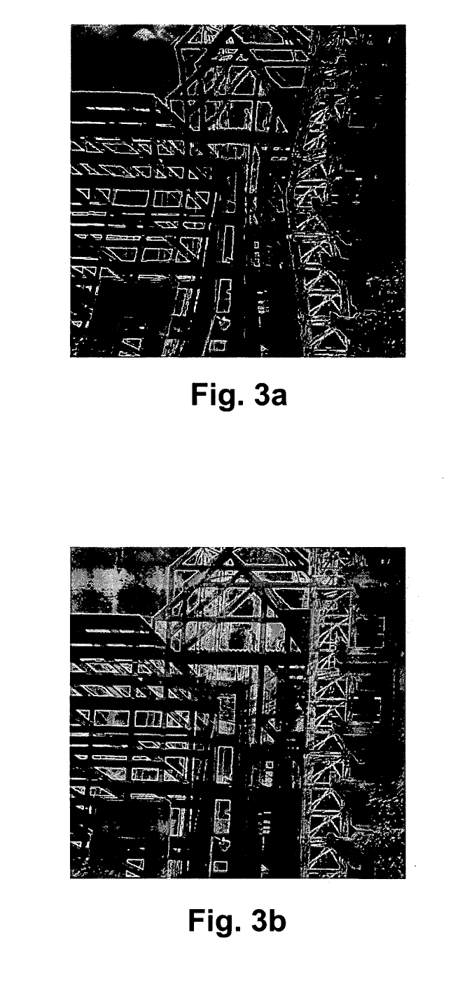

1) Measure the retinal warp of the patient. The result is a 2D warped grid representing the complement or inverse of the physical distortions in the patient's retina. This process is depicted in FIG. 2a, and is monocularly represented in schematic form in FIG. 2b.
   Reconstruct the Patient's World
1) Orient Observer with respect to Scene Geometry.
We start with the Scene Geometry in World Space. The Observer is located near the Scene Geometry and is oriented toward the geometry such that the Scene Geometry is within the Observer's cameras field-of-view. Both camera views center on the same 3D location. The orientation of each camera will be slightly different due to the interocular separation.
2) Triangulate the Scene Geometry.
We triangulate the Scene Geometry in order to add density. All higher order polygons are reduced to triangles. These triangles are further subdivided to create more 3D geometry points. This creates more data to warp and results in a smoother resulting geometry.
3) Project Scene Geometry onto Observer's Retinal Planes.
We project the 3D points in the Scene Geometry toward each Eye Nodal point onto each Retina Image Plane. Thus, each 3D point in the Scene Geometry has a pair of corresponding 2D point located in the two Retina Image Planes.
4) Warp Data Points in the Retina Image Plane.
The 2D points in the Retina Image Planes are warped by the documented inverse retina distortions. This moves the 2D points to the location where the patient perceives the point.
5) Project Data Points back out into World Space.
For every pair of corresponding 2D data points (see 3 above), A ray is cast from the Eye Nodal Point, through the data point into World Space. There are two rays, one for each 2D data point and Eye Nodal Point pair.
On each ray, the point of closest approach, Pleft and Pright, of the two rays is determined. Dy is defined as the vertical distance between Pleft and Pright, as viewed by the Observer.
6) Generate Left Eye Geometry and Right Eye Geometry.
Through analysis of Pleft, Pright, and Dy we generate the Left Eye Geometry and Right Eye Geometry. The two geometries have the same geometric structure as the original Scene Geometry, however the specific location of the actual 3D data points are perturbed according to the followings function:
Where: Pleft is the point of closest approach on the left ray Pright is the point of closest approach on the right ray Dy is the distance between Pleft and Pright Ey is the allowable vertical separation of Pleft and Pright before stereo fusion fails.
if Dy<Ey then P=Midpoint(Pleft->Pright) set P as the location of the corresponding point in the Left Eye Geometry and Right Eye Geometry
otherwise, set Pleft as the location of the corresponding point the Left Eye Geometry, and set Pright as the location of the corresponding point the Right Eye Geometry
7) Render Reconstructed Image
We now have 2 warped geometries where some of the corresponding points are collocated in the same 3D position and some of the corresponding points are separated. We render these 2 geometries with variable (preferably 50%) transparency. The collocated points will combine to form a 100% solid structure. The separated points will create a double-image effect. Gross distortions in the geometry arising from stereo reconstruction error due to the warped data points is also apparent. This effect is illustrated in FIGS. 3a, and 3b.
The user may rotate the reconstructed geometry to inspect the distortions from positions other that the Observer's position.
The algorithm described above is only one embodiment of the present invention. Other stereo reconstruction algorithms may also be employed with good results. Such other algorithms may include:
Warp not only the data points but the geometry itself.
Warp the world space in which the geometry lies.
Reconstruct the patient world view based on image-based rendering techniques, such as image stereopsis.
The method of the present invention is demonstrated in the source code of the accompanying Appendix, which is © 1998-9 Sinclair Vision, Inc. and may be reproduced solely for purposes of reproduction of any patent that issues from this application.

The method of the present invention is being implemented in a limited form in a self-evaluation site on the World Wide Web, and on other computer systems and networks. The following is a detailed explanation of the evaluation to be conducted by users of the web site.

### Clear Vision Website Vision Testing Section

Purpose of Testing in this Website:

1) To screen for vision abnormalities in the public sector, independent of eye-care practitioners' offices;
2) To gather data of public vision screening test abnormalities and the correlation with age and with perceived abnormalities of vision and the effect on tasks of daily living
3) To acquaint the public and eye-care practitioners of these methods for testing persons with ocular pathology.
   Specifications:
   1. All letters at least similar to font size Times 20.
   2. Website should be available to most all browsers
   3. Website should allow both text to speech (e.g. see www.manasota.com/blindrap/index.html, uses Talker 2.0 from MVP for MAC) and should (2nd priority) during testing present commands with audio streaming (e.g. see MacWorld February 1998, page 125 or www.macworld.com)
   4. At the bottom of each page (except during test) should be pointers to Clear Vision Home page, Testing Section (points to Introduction and Demographics Page), Vision Testing Results, The Eye and Vision, Vision Abnormalities
      Protocol:
      Introduction and Demographics Page:
The following statement should appear on the Introduction and Demographics page of the Clear Vision Website:
“The vision testing in this website is provided to the public without charge by a generous donation from Sinclair Vision, Inc.
Server: This website provides without charge a series of tests for your vision. You also will be asked questions about whether you feel you have vision problems and how severely those problems interfere with your daily activities. While this testing is intended to discover possible vision abnormalities, and we encourage you to share the results with your optometrist or ophthalmologist, this test IS NOT A substitute for a complete ocular examination provided by a professional.

To take the test you must first provide your name, your age, and a pass-word along with a pass-number. The answers you provide and the results of this testing of your vision will be kept confidential and will not be released by us to anyone unless your name, your pass-word and pass-number (which you will enter below) are supplied. We suggest that you write down the pass-word and pass-number since this website provides many different vision tests that you may wish to take at different times:

First Name ______

Last Name ______

Pass-word ______ (must be at least 6 letters and/or numbers)

Pass-number ______ (must be at least 6 digits)

Age ______ (required to compare your results with others of the same age)

Have you taken a vision test at this website before? no_ yes_{—}
If yes, do you wish to:
   Get the results of tests taken previously? _{—}
   Take a new test? _{—}
   Take again a previously taken test? _{—}
   Answer again the survey questions about vision problems? _{—}
Client: uploads data to server which compares against data base to determine, if client has not registered before, that the combination uploaded of name, pass-word, and pass-number are unique (also need to consider uniqueness for fax-on-demand push-button telephone). ?Consideration: Should the server repeat the above data back to the client (or make the client repeat the pass-word and pass-number) for verification?
If client has not registered before and registration information is not unique in data base:
If client indicates he wishes to get results of tests taken previously:

Server: down loads results of the survey questions answered previously and tests taken previously
If client indicates he wishes to take a new test:

Server: Searches data base of questions and tests taken:

Introduction to Testing Page

We will now conduct a test of your vision and will test each eye separately. The accuracy of the test depends upon your following these guidelines as carefully as possible. To take the test you will need a piece of string, a ruler, and 3 small pieces of tape:
1. If you normally look at your monitor with glasses or contact lenses, please take the tests with the glasses or contact lenses on. If you use bifocal glasses, please tape them up to your forehead with one of the small pieces of tape so that you can see the entire monitor well while looking at it's center.
2. Please measure the following lines using a ruler.
(On the screen vertical and horizontal lines are presented with space to type the length in inches or cm.)
3. If this computer supports audio do you want the instructions presented verbally?

yes_ no_{—}
4. (A bar is presented on screen with 4 sections: 100% dark, 98% dark, 100% white and 98% white). You will now adjust the contrast and illumination of the monitor screen. Sit comfortably in front of the monitor in as dark a room as possible (Taking the test at night is even better). Put the contrast control on the monitor up to maximum and turn the brightness control until you see the black bar as black as as possible but while you can still see 2 distinct bars. If you have poor vision please ask someone to help you make the adjustment.
5. You must sit 24 inches (60 cm) from the monitor. You can simply sit with your arms outstretched and with your outstretched fingers barely touching the monitor. For more accurate results you can tie 2 knots in a string 24 inches (60 cm) apart. Tape one knot to the top of the monitor screen and sit so that your forehead just touches the other knot.
6. You must be able to easily reach the cursor keys (arrow keys) on the keyboard. Position your fingers over the right and left (forward and backward) cursor keys such that you can press them without looking directly at the keys.
7. We will test one eye at a time. We would suggest that you first test the eye with the better vision. You can tape the other eyelid closed with a small piece of tape by touching one end to the upper lid and pulling the lid down, stick the other end of the tape to your cheek. Which eye do you wish to test first?

Right eye_{—}

Left Eye_{—}
Client uploads the screen dimensions, audio preferences, and eye to be tested first.
Server downloads the practice test page.
   Practice Test Page

A black screen is presented with a cross in the center that is pink or orange (=50% gray if monitor does not support color). The directions will appear across the top of the screen in at least Times 20 font or larger, or if the computer has sound the instructions can be presented with audio streaming.
“Always keep your vision directed at the center of the cross. At the center of the cross a letter C will appear with the opening facing to the right or to the left”
A C measuring 100 mins of arc appears at the center of the cross, white against a black pedestal twice the size of the letter. The opening of the C faces to the left. The C remains on the screen until a cursor key is depressed and the appropriate response is shown or read.
“Please press now the cursor key of the direction you think the opening of the C is facing”
If the left cursor key is depressed: “Correct. The opening of the C was facing to the left.
If the right cursor key is depressed: “No, that is not correct. The opening of the C was facing to the left. The large C then disappears to be replaced another 200 mins arc C facing to the right and the same process is repeated. If the person fails again to identify the correct orientation, the testing of the eye is aborted.
If a correct response is obtained, the C disappears and the instruction appears “The C will now flash on the screen for only a short time.”
A 100 min arc C flashes on the screen facing to the right for one second.
“As soon as you can, press the left or right cursor keys depending upon which way you think the C was facing.”
If the right cursor key is depressed: “Correct. The opening of the C was facing to the right.
If the left cursor key is depressed: “No, that is not correct. The opening of the C was facing to the right. The large C then disappears to be replaced another 200 mins arc C facing to the left and the same process is repeated. If the person fails again to identify the correct orientation, the testing of the eye is aborted.
If a correct response is obtained, the C disappears and the instruction appears “Keep your hand over the cursor keys so that you can respond as quickly as possible without looking directly at the keys.”
A 100 min arc C flashes on the screen facing to the right for one-half second.
“As soon as you can, press the left or right cursor keys depending upon which way you think the C was facing.”
If the right cursor key is depressed: “Correct. The opening of the C was facing to the right.
If the left cursor key is depressed: “No, that is not correct. The opening of the C was facing to the left. The large C then disappears to be replaced another 200 mins arc C facing to the left and the same process is repeated. If the person fails again to identify the correct orientation, the testing of the eye is aborted.
If correct, the process is again repeated with the 100 min arc C presented for 250 msec if age<60, or for 330 msec if age≧60.
If a correct response is given: “Correct, the C flashed quickly, but you correctly identified that it was facing to the left . . . . In the test that follows, you will have about 1 second after the C flashes to respond. Please press the appropriate cursor key as soon after the C flashes as you can. The faster you respond, the faster the test will go. The C will vary in size and contrast; some will be a white C against a black background; some will be a black C against a white background. We will now proceed with the testing of your ______ eye.”
Server: Downloads to client the screening vision test consisting of:

High Contrast Acuity: a white, high contrast C is presented centrally against a black background)—test starts at 50 min arc

Low contrast Acuity: (90% black) 90% gray-black C presented centrally against a 100% black background.

Low contrast Acuity with glare: 90% white letter presented centrally against a 100% white pedestal and white background.

At the end of the testing the client computer asks whether the person wishes to test the other eye and then reiterates the testing procedure.
Client: Uploads to server the test results page for both eyes. Before he uploads the results the following message should appear:
The results of this testing of your vision will be kept confidential and will not be released by us to anyone unless your name, your pass-word and pass-number (which you have entered) are supplied. While this testing is intended to discover possible vision abnormalities and we encourage you to share the results with your optometrist or ophthalmologist, this test is not a substitute for a complete ocular examination provided by a professional.
Server: Downloads to client the results (e.g. 20/30 High Contrast Acuity, 20/50 acuity at 10% contrast, 20/100 acuity at 10% contrast with glare) together with 3 pictures altered according to the test data obtained (for each eye) and a paragraph containing the age and results of answers to questions together with the following message printed at the top of the page:
“The testing and the results at this website are provided to the public without charge by a generous grant from ______ and no attempt at collection of payment is allowed.” and the following message at the bottom of the page:
“The tests results, compared with those of persons your same age appear to be normal (abnormal), but this website cannot control the conditions of the testing. We would recommend that, if you wish, you share these results with your medical doctor or eye doctor:

1) You may print the results if you have a printer:
On a IBM PC by ______
On a Macintosh by ______ or

2) You or your physician may obtain the results by fax or by mail by dialing ______ (the name, pass-word, and pass-number must be supplied).”

Server: downloads the introduction to distortion testing module.
### We will now conduct another test of your vision and will test each eye separately. The accuracy of the test depends upon your following these guidelines as carefully as possible. To take the test you will need a piece of string, a ruler, and 3 small pieces of tape:
1. If you normally look at your monitor with glasses or contact lenses, please take the tests with the glasses or contact lenses on. If you use bifocal glasses, please tape them up to your forehead with one of the small pieces of tape so that you can see the entire monitor well while looking at it's center.
2. Please measure the following lines using a ruler.
(On the screen vertical and horizontal lines are presented with space to type the length in inches or cm.)
3. If this computer supports audio do you want the instructions presented verbally?

yes_ no_{—}
4. (A bar is presented on screen with 4 sections: 100% dark, 98% dark, 100% white and 98% white). You will now adjust the contrast and illumination of the monitor screen. Sit comfortably in front of the monitor in as dark a room as possible (Taking the test at night is even better). Put the contrast control on the monitor up to maximum and turn the brightness control until you see the black bar as black as as possible but while you can still see 2 distinct bars. If you have poor vision please ask someone to help you make the adjustment.
5. You must sit 24 inches (60 cm) from the monitor. You can simply sit with your arms outstretched and with your outstretched fingers barely touching the monitor. For more accurate results you can tie 2 knots in a string 24 inches (60 cm) apart. Tape one knot to the top of the monitor screen and sit so that your forehead just touches the other knot.
6. You must be able to easily reach the cursor keys (arrow keys) on the keyboard or your mouse or other pointing device. Position your fingers over the cursor keys such that you can press them without looking directly at the keys, or grasp your mouse or other pointing device comfortably.
7. We will test one eye at a time. We would suggest that you first test the eye with the better vision. You can tape the other eyelid closed with a small piece of tape by touching one end to the upper lid and pulling the lid down, stick the other end of the tape to your cheek. Which eye do you wish to test first?

Right eye_{—}

Left Eye_{—}
Client uploads the screen dimensions, audio preferences, and eye to be tested first.
Server downloads the spline grid, and the Client manipulates the grid to remove perceived distortions.
If no abnormal results:

Server: downloads to client High contrast B/W Central Field module If client provided abnormal results to questions other than yes to distortions:
Server downloads to client High contrast B/W Central Field module If client has answered yes to any of distortion or diplopia (double vision) questions:
Server: downloads distortion module
If client indicates he wishes to retake survey questions about vision problems:

Server: downloads to client survey questions about vision problems; when answers are again uploaded, should the new data replace previous answers or should both answer sets be stored (what if client wishes to take same test, or answer questions and maintain the results from different times, i.e. before and after cataract surgery)
If client wishes to retake a previously taken test::

Server: A search of our database indicates you have previously taken the following tests. Which one do you wish to take again? (Consider using icons with subtitles for each testing module)
Screening test of central vision _{—}
Central field, high contrast targets _{—}
Central field distortions _{—}

Client: Uploads choice of desired test

Server: Downloads desired test (consider ? what if client wishes to take a test again, but maintain answers from the same test taken previously, (i.e. before and after cataract surgery)
Survey: General Questions about Vision Problems Page:
Server: We will first ask a few questions about vision problems that may be bothering you and may limit some of your activities. If you wear glasses or contact lenses for a particular activity, please answer the question as if you were wearing the glasses or contact lenses.
24. At the present time, would you say your vision at distance (with glasses or contact lenses, if you wear them) using both eyes is:

Excellent_{—}

Good_{—}

Fair_{—}

Poor_{—}

Very Poor_{—}

Completely Blind_{—}
23. How much difficulty do you have reading ordinary print in newspapers?

No difficulty_{—}

A little difficulty_{—}

Moderate difficulty_{—}

Extreme difficulty_{—}

Stopped reading because of eyesight_{—}

Stopped reading for other reasons or not interested_{—}
22. Because of your eyesight, how much difficulty do you have figuring out whether bills you receive are accurate?

No difficulty_{—}

A little difficulty_{—}

Moderate difficulty_{—}

Extreme difficulty_{—}

Stopped doing because of eyesight_{—}

Stopped doing for other reasons or not interested_{—}
21. How much difficulty do you have with work or hobbies that require you to see well up close (for example cooking, sewing, or fixing things using hand tools)?

No difficulty_{—}

A little difficulty_{—}

Moderate difficulty_{—}

Extreme difficulty_{—}

Stopped doing because of eyesight_{—}

Stopped doing for other reasons or not interested_{—}
20. Because of eyesight, how much difficulty do you have finding something on a crowded shelf, for example in a store?

No difficulty_{—}

A little difficulty_{—}

Moderate difficulty_{—}

Extreme difficulty_{—}
19. At a distance how much difficulty do you have reading street signs or the names of stores on signs?

No difficulty_{—}

A little difficulty_{—}

Moderate difficulty_{—}

Extreme difficulty_{—}
18. Because of your eyesight, how much difficulty do you have taking part in outdoor sports you enjoy?

No difficulty_{—}

A little difficulty_{—}

Moderate difficulty_{—}

Extreme difficulty_{—}

Stopped doing because of eyesight_{—}

Stopped doing for other reasons or not interested_{—}
17. Because of your eyesight, how much difficulty do you have recognizing people you know across a room or across the street?

No difficulty_{—}

A little difficulty_{—}

Moderate difficulty_{—}

Extreme difficulty_{—}
16. Because of your eyesight, how much difficulty do you have seeing and enjoying programs on TV?

No difficulty_{—}

A little difficulty_{—}

Moderate difficulty_{—}

Extreme difficulty_{—}

Stopped doing because of eyesight_{—}

Stopped doing for other reasons or not interested_{—}
15. Because of your eyesight, how much difficulty do you have going down steps, stairs, or curbs in dim light?

No difficulty_{—}

A little difficulty_{—}

Moderate difficulty_{—}

Extreme difficulty_{—}

Stopped doing because of eyesight_{—}

Stopped doing for other reasons or not interested_{—}
14. Because of your eyesight, how much difficulty do you have noticing objects off to the side or bumping into objects while you are walking along?

No difficulty_{—}

A little difficulty_{—}

Moderate difficulty_{—}

Extreme difficulty_{—}

Stopped doing because of eyesight_{—}

Stopped doing for other reasons or not interested_{—}
13. Because of your eyesight, how much difficulty do you have seeing how people react to things you say?

No difficulty_{—}

A little difficulty_{—}

Moderate difficulty_{—}

Extreme difficulty_{—}
12. Because of your eyesight, how much difficulty do you have visiting with people in their homes, at parties, or in restaurants?

No difficulty_{—}

A little difficulty_{—}

Moderate difficulty_{—}

Extreme difficulty_{—}

Stopped doing because of eyesight_{—}

Stopped doing for other reasons or not interested_{—}
11. Because of your eyesight, how much difficulty do you have going out to see movies, plays, or sports events?

No difficulty_{—}

A little difficulty_{—}

Moderate difficulty_{—}

Extreme difficulty_{—}

Stopped doing because of eyesight_{—}

Stopped doing for other reasons or not interested_{—}
10. Do you currently drive a car, at least once in a while?

yes_{—}

No_{—}
9. If no, have you never driven a car or have you given up driving?

Never have driven_{—}

Gave up driving_{—}
8. If you gave up driving, was the main reason your eyesight, or mainly for some other reason, or because of both your eyesight and other reasons?

Mainly eyesight_{—}

Mainly other reasons_{—}

Both eyesight and other reasons_{—}
7. If you currently are driving, How much difficulty do you have driving during the daytime?

No difficulty_{—}

A little difficulty_{—}

Moderate difficulty_{—}

Extreme difficulty_{—}
6. How much difficulty do you have driving at night?

No difficulty_{—}

A little difficulty_{—}

Moderate difficulty_{—}

Extreme difficulty_{—}
5. Do you accomplish less than you would like because of your vision?

All of the time_{—}

Most of the time_{—}

Some of the time_{—}

A little of the time_{—}

None of the time_{—}
4. Are you limited in how long you can work or do other activities because of your vision?

All of the time_{—}

Most of the time_{—}

Some of the time_{—}

A little of the time_{—}

None of the time_{—}
3. Are you afraid to go out alone because of your eyesight?

All of the time_{—}

Most of the time_{—}

Some of the time_{—}

A little of the time_{—}

None of the time_{—}
2. Do you feel frustrated because of your eyesight?

All of the time_{—}

Most of the time_{—}

Some of the time_{—}

A little of the time_{—}

None of the time_{—}
1. Do you feel you have to rely too much on others because of your eyesight?

All of the time_{—}

Most of the time_{—}

Some of the time_{—}

A little of the time_{—}

None of the time_{—}
Survey: Questions about Particular Vision Problems Page
Server: We wish now to ask some brief questions about particular problems you may experience with your vision. Start by clicking on one or more of the items below that might be troubling you:

(Using picture icons for each problem that user can click on)

_Reduced vision in one eye—The vision in one eye is much more severely reduced than in the other eye

_Glare—Halos or sparkles are seen around lights at night, or severe glare is observed that washes out your vision on a sunny day (if you forgot your sunglasses).

_Double vision—Two images are seen or ghosting of the image.

_Distortions—Crooked or wavy parts of your vision are noted when you look at straight objects (for example a window, a doorway, or printed material).

_Defects in the field of vision—Parts of your vision are blurred, washed out, or the color is altered.

_Reduced vision under dim lighting—In a restaurant or theater it takes a long time for your eyes to adjust or they never adjust enough to allow you to see clearly at distance.

_Floaters—Dots, rings, or cobwebs are seen floating in the vision of one or both eyes.

_Excessive burning, irritation or tearing—After periods of visual concentration (e.g. reading, driving, or watching television) your eyes burn, are irritated, or tear excessively
Client: Submits Data from General Survey Questions and Questions of Particular Vision Problems pages to Server:

Server downloads a page of further questions for each of the items that were checked:

If Reduced Vision in One Eye is indicated by client server downloads Reduced vision in one eye page.

Reduced Vision in One Eye Page

Server: (use the picture icon) You have indicated that one eye has much more severely reduce vision than the other eye.
1. Which eye has the more severely reduced vision?

Right eye_{—}

Left Eye_{—}
2. Has the vision been reduced since your were a child?

Yes_{—}

No_{—}
3. Does the reduced vision in one eye cause you any difficulty reading with both eyes (and using your reading glasses, if you usually wear them)?

No difficulty_{—}

A little difficulty_{—}

Moderate difficulty_{—}

Extreme difficulty_{—}

Stopped reading because of vision problems_{—}

Stopped reading for other reasons or not interested_{—}
4. Does the reduced vision in one eye cause difficulty with driving?

No difficulty_{—}

A little difficulty_{—}

Moderate difficulty_{—}

Extreme difficulty_{—}

Stopped driving because of vision problems_{—}

Stopped driving for other reasons or have never driven_{—}
5. Does the reduced vision in one eye cause difficulty with work or hobbies that require you to see well up close (for example cooking, sewing, or fixing things using hand tools)?

No difficulty_{—}

A little difficulty_{—}

Moderate difficulty_{—}

Extreme difficulty_{—}

Stopped doing because of vision problems_{—}

Stopped doing for other reasons or not interested_{—}

If Glare is indicated by client, server downloads Glare page:

Glare Page

Server: (use picture icon) You indicated that glare causes you vision problems. Glare depends upon the lighting conditions and often will cause haziness when the light is shining in your eyes. If you wear glasses or contact lenses, please answer the questions as if you were wearing them (but not sunglasses).
1. On a sunny day does glare cause difficulty driving without sunglasses (or glasses with a significant tint)?

No difficulty_{—}

A little difficulty_{—}

Moderate difficulty_{—}

Extreme difficulty_{—}

Stopped driving because of vision problems_{—}

Stopped driving for other reasons or never drove_{—}
2. If you prefer to wear sunglasses, do they help with glare while driving?

Provide no help_{—}

Provide some help_{—}

Provide great help_{—}

Stopped driving because of vision problems_{—}

Stopped driving for other reasons or have never driven_{—}
3. On a sunny day does glare cause difficulty for you to participate in your favorite outdoor sport or activity (without sunglasses)?

No difficulty_{—}

A little difficulty_{—}

Moderate difficulty_{—}

Extreme difficulty_{—}
4. If you prefer to wear sunglasses, do they help with your favorite outdoor activity?

Provide no help_{—}

Provide some help_{—}

Provide great help_{—}
5. Does glare from the headlights of oncoming cars cause difficulty with driving at night?

No difficulty_{—}

A little difficulty_{—}

Moderate difficulty_{—}

Extreme difficulty_{—}

Stopped driving because of vision problems_{—}

Stopped driving for other reasons or have never driven_{—}
6. Does glare cause difficulty with reading or limit your reading?

No difficulty_{—}

A little difficulty_{—}

Moderate difficulty_{—}

Extreme difficulty_{—}

Don't read_{—}
7. Glare problems are often worse in one eye. Do you feel the glare problems described above:

In the Right eye are:

None_{—}

Minimal_{—}

Moderate_{—}

Severe_{—}

In the Left eye are:

None_{—}

Minimal_{—}

Moderate_{—}

Severe_{—}

If Double vision is indicated by client, server downloads Double vision page:

Double Vision Page

Server: (use picture icon) You have indicated that you notice double vision (or ghosting of the image) in your vision.

We must first determine whether this is caused by both eyes seeing two different images or whether one eye alone is causing the double image:
Test 1: Look at something that produces your double vision (perhaps while looking at television or while reading). Wear your glasses if you wear them for distance or your reading glasses if the double image bothers you while reading. First cover the right eye (using your hand—no cheating!) then uncover and now cover the left eye. Does the double image disappear when either eye is covered?

_Yes: The double vision is seen only when both eyes are open, but disappears if either eye is covered

_No: The double vision is still seen when one eye is covered
Which eye still sees the ghosted or double image when the other is covered?

Right eye_{—}

Left eye_{—}
If the double or ghosted image is seen only with both eyes open, this indicates that the brain is being confused by different images coming from each eye. For example the image from one eye is bigger or smaller than the image from the other eye, or the image from one eye is distorted (crooked) and the brain can't line things up with both eyes, or each eye is looking at a different object (the eyes are not lined up).
Test 2: Cover each eye as you did above to compare the image seen from the right eye with that seen with the left eye.
2A. Does the image from one eye look smaller (or further away) than the image from the other eye?

yes_{—}

no_{—}
2B. If the image from one eye is smaller. Which eye sees the smaller image?

Right eye_{—}

Left eye_{—}
2C. How much smaller is the image than the image in the other eye?

10% smaller_{—}

20% smaller_{—}

30% smaller_{—}

more than 30% smaller_{—}
3A. Is the image from one eye crooked (or taller or fatter) than the other eye's image?

yes_{—}

no_{—}
3B. Which eye do you believe has the distorted or crooked image?

Right eye_{—}

Left eye_{—}
4. Does the double vision cause difficulty with reading or limit your reading (with both eyes open)?

No difficulty_{—}

A little difficulty_{—}

Moderate difficulty_{—}

Extreme difficulty_{—}

Stopped reading because of vision problems_{—}

Stopped reading for other reasons or not interested in reading_{—}
5. Does the double vision cause difficulty with driving?

No difficulty_{—}

A little difficulty_{—}

Moderate difficulty_{—}

Extreme difficulty_{—}

Stopped driving because of vision problems_{—}

Stopped driving for other reasons or have never driven_{—}
6. Does the double vision cause difficulty with work or hobbies that require you to see well up close (for example cooking, sewing, or fixing things using hand tools)?

No difficulty_{—}

A little difficulty_{—}

Moderate difficulty_{—}

Extreme difficulty_{—}

Stopped doing because of vision problems_{—}

Stopped doing for other reasons or not interested_{—}

If Distorted vision is indicated, the server downloads the Distortion Page:

Distortion Page

Server: (use picture icon) You have indicated that you have distorted or crooked vision (or kinks in straight lines of the vision). Usually this is present in only one eye.
Test 1: Look at something that produces the distortion (perhaps while looking at a window, at television, or while reading). Wear your glasses if you wear them for distance or your reading glasses if the crooked image bothers you while reading. First cover the right eye (using your hand—no cheating!) then uncover and now cover the left eye.
1A. Which eye has the distorted vision?

Right eye_{—}

Left eye_{—}
Often in the eye with distorted or crooked vision, the image is smaller or bigger than the image from the other eye.
Test 2: Do the same test again (alternately covering each eye while looking at the window, television, or reading material) but this time compare the size of the image in the eye with the distorted vision with the size of the image in the other eye.
2A. Is the image that is distorted also smaller or bigger than the image in the other eye?

Smaller (objects look further away)_{—}

Bigger (objects look closer)_{—}

No difference in size_{—}
3. Does the distorted vision cause difficulty with reading or limit your reading?

No difficulty_{—}

A little difficulty_{—}

Moderate difficulty_{—}

Extreme difficulty_{—}

Stopped reading because of vision problems_{—}

Stopped reading for other reasons or not interested in reading_{—}
4. Does the distorted vision cause difficulty with driving?

No difficulty_{—}

A little difficulty_{—}

Moderate difficulty_{—}

Extreme difficulty_{—}

Stopped driving because of vision problems_{—}

Stopped driving for other reasons or have never driven_{—}
5. Does the distorted vision cause difficulty with work or hobbies that require you to see well up close (for example cooking, sewing, or fixing things using hand tools)?

No difficulty_{—}

A little difficulty_{—}

Moderate difficulty_{—}

Extreme difficulty_{—}

Stopped doing because of vision problems_{—}

Stopped doing for other reasons or not interested_{—}
If Defects in the Field of Vison is indicated by client, server downloads Defects in the field of vision Page:
   Defects in the Field of Vision Page

Server: (use picture icon) You have indicated that you have defects (smudges, gray areas or areas of faded color or less contrast in the visual field of one or both eyes.
Right Eye:
R1. Do you have visual field defects in your right eye?

yes_{—}

no_{—}
R2. If yes, do the visual field defects involve the center of the vision in your right eye?

yes_{—}

no_{—}
R3. Do the visual field defects involve the peripheral vision of the right eye?

yes_{—}

no_{—}
R4. Do the field defects cause difficulty with reading or limit your reading?

No difficulty_{—}

A little difficulty_{—}

Moderate difficulty_{—}

Extreme difficulty_{—}

Stopped reading because of vision problems_{—}

Stopped reading for other reasons or not interested in reading_{—}
R5. Do the field defects cause difficulty with driving?

No difficulty_{—}

A little difficulty_{—}

Moderate difficulty_{—}

Extreme difficulty_{—}

Stopped driving because of vision problems_{—}

Stopped driving for other reasons or have never driven_{—}
R6. Do the field defects cause difficulty with work or hobbies that require you to see well up close (for example cooking, sewing, or fixing things using hand tools)?

No difficulty_{—}

A little difficulty_{—}

Moderate difficulty_{—}

Extreme difficulty_{—}

Stopped doing because of vision problems_{—}

Stopped doing for other reasons or not interested_{—}
Left Eye:
L1. Do you have visual field defects in your right eye?

yes_{—}

no_{—}
L2. If yes, do the visual field defects involve the center of the vision in your right eye?

yes_{—}

no_{—}
L3. Do the visual field defects involve the peripheral vision of the left eye?

yes_{—}

no_{—}
L4. Do the field defects cause difficulty with reading or limit your reading?

No difficulty_{—}

A little difficulty_{—}

Moderate difficulty_{—}

Extreme difficulty_{—}

Stopped reading because of vision problems_{—}

Stopped reading for other reasons or not interested in reading_{—}
L5. Do the field defects cause difficulty with driving?

No difficulty_{—}

A little difficulty_{—}

Moderate difficulty_{—}

Extreme difficulty_{—}

Stopped driving because of vision problems_{—}

Stopped driving for other reasons or have never driven_{—}
L6. Do the field defects cause difficulty with work or hobbies that require you to see well up close (for example cooking, sewing, or fixing things using hand tools)?

No difficulty_{—}

A little difficulty_{—}

Moderate difficulty_{—}

Extreme difficulty_{—}

Stopped doing because of vision problems_{—}

Stopped doing for other reasons or not interested_{—}
If Reduced Vision under dim lighting conditions is indicated by the client: Server downloads Reduced vision under dim lighting conditions page
   Reduced Vision Under Dim Lighting Conditions Page

Server: (Use picture icon) You have indicated that your vision is reduced under conditions of dim lighting.
1. In a restaurant, after a half hour, how much difficulty do you have reading the menu (with your reading glasses if you use them)?

No difficulty_{—}

A little difficulty_{—}

Moderate difficulty_{—}

Extreme difficulty_{—}

Stopped doing because of vision problems_{—}

Stopped doing for other reasons or not interested in reading the menus_{—}
2. In a restaurant or a theater, after a half hour, how much difficulty do you have recognizing faces across the room?

No difficulty_{—}

A little difficulty_{—}

Moderate difficulty_{—}

Extreme difficulty_{—}
3. Does the reduced vision cause difficulty when driving at night on streets that are well lighted?

No difficulty_{—}

A little difficulty_{—}

Moderate difficulty_{—}

Extreme difficulty_{—}

I Don't drive at night_{—}
4. Does the reduced vision in dim lighting cause problems driving at night on poorly lit streets?

No difficulty_{—}

A little difficulty_{—}

Moderate difficulty_{—}

Extreme difficulty_{—}

I Don't Drive at night_{—}
If floaters are indicated by the client: Server downloads to client Floaters Page:
   Floaters Page

Server: (Use picture icon) You have indicated that you have floaters (spots, rings, or cobwebs) that float in the vision of one or both eyes even when you hold your eyes still.
1. Which eye has the worst floaters

Right eye_{—}

Left eye_{—}
2. Do the floaters cause difficulty with reading or limit your reading?

No difficulty_{—}

A little difficulty_{—}

Moderate difficulty_{—}

Extreme difficulty_{—}

Stopped reading because of floaters_{—}

Stopped reading for other reasons or not interested in reading_{—}
3. Do the floaters cause difficulty with driving?

No difficulty_{—}

A little difficulty_{—}

Moderate difficulty_{—}

Extreme difficulty_{—}

Stopped driving because of floaters problems_{—}

Stopped driving for other reasons or have never driven_{—}
4. Do the floaters cause difficulty with work or hobbies that require you to see well up close (for example cooking, sewing, or fixing things using hand tools)?

No difficulty_{—}

A little difficulty_{—}

Moderate difficulty_{—}

Extreme difficulty_{—}

Stopped doing because of floaters_{—}

Stopped doing for other reasons or not interested_{—}
If the client indicates he suffers Ocular irritation, burning or excessive tearing:

Server Downloads Ocular irritation, burning or excessive tearing page

Ocular Irritation, Burning or Excessive Tearing Page

Server: You have indicated that after periods of visual concentration (e.g. reading, driving, or watching television) your eyes burn, are irritated, or tear excessively
1. How severe would you consider the burning, itching, aching or excessive tearing?

Mild_{—}

Moderate_{—}

Severe_{—}

Very Severe_{—}
2. Does the burning or tearing keep you from doing what you'd like to be doing?

All of the time_{—}

Most of the time_{—}

Some of the time_{—}

A little of the time_{—}

None of the time_{—}
3. Does the burning or tearing cause difficulty with reading or limit your reading?

No difficulty_{—}

A little difficulty_{—}

Moderate difficulty_{—}

Extreme difficulty_{—}

Stopped reading altogether because of burning or tearing_{—}

Stopped reading for other reasons or not interested in reading_{—}
4. Does the burning or tearing cause difficulty with work or hobbies that require you to see well up close (for example cooking, sewing, or fixing things using hand tools)?

No difficulty_{—}

A little difficulty_{—}

Moderate difficulty_{—}

Extreme difficulty_{—}

Stopped doing because of irritation_{—}

Stopped doing for other reasons or not interested_{—}

Client: uploads to Server answered questions data from the Page(s) that had been downloaded:

Server: Reminder: all information supplied at this website is confidential. It will be released only if you supply the name along with the pass-word and pass-number. Do not give these out unless you want that person to access your information. We suggest you write down your pass-word and pass-number for future reference.

Reduced Vision in one eye page

Glare page

Double Vision page

Distortion page

Reduced vision under dim illumination page

Floaters page

Ocular irritation page
Server: Downloads to Client the Introduction to Testing Page
Server: Thank you for spending time at this website; we hope it has been of service. This website offers other tests of your vision. Do you wish to take any further tests?
yes_=Return to Introduction and Demographics page_{—}
no_ Goodby
The Multi-Axis Vision Evaluation System, utilized to measure vision abnormalities in this website, is patented in the United States with patents pending in Canada, the UK, Germany, and Israel. Any attempts to commercially market this or a similar vision measurement system without the expressed permission of the Clear Vision Foundation represent an infringement of such patents and will be prosecuted to the fullest. In summary, the Clear Vision Foundation grants a non-transferable, personal license to use the MAVE-Web software for internal purposes only. You may not use the data or program for the development of data related products or services, disclose any software or data or portion thereof, to any third party (including your affiliates), or copy any software or data without the expressed, written permission of the Clear Vision Foundation.

The present invention has been described herein with respect to particular implementations and embodiments, which are intended to teach and illustrate the invention, and not as limitations of the spirit of the present invention, which are solely embodied in the claims hereof.

### STATEMENT OF INDUSTRIAL UTILITY

The present invention may be employed to test and quantify a visual defect known as metamorphopsia, and to present to other observers the perceived world-view of one suffering from this condition.

We claim as our invention:

1. A method for quantifying the visual distortions characteristic of metamorphopsia, comprising the steps of:
a. Measuring the retinal warp of each eye of a human subject;
b. Orienting an observer with a scene geometry in a world space;
c. Triangulating scene geometry;
d. Projecting the triangulated scene geometry on to the observer's retinal planes;
e. Warping projected scene geometry data points in the retinal planes by the measured inverse retinal warp from step (a);
f. Projecting scene geometry data points back into the world space;
g. Generating eye geometries for each eye of the human subject;
h. Rendering a reconstructed image from the eye geometries, simulating the binocular perception of the human subject by combining each eye to account for measured or assumed eye dominance.

2. The system of claim 1 wherein said human subject's retinal warp is tested using a site on the internet.

3. The system of claim 1 wherein said reconstructed image is reconstructed in real time in response to movement of the human subject's point of visual fixation and head.
